(19) United States
(12) Patent Application Publication
(10) Pub. No.: US 2025/0108171 A1
(43) Pub. Date: Apr. 3, 2025

(54) DOSE MONITORING SYSTEM FOR INJECTION DEVICE

(71) Applicant: Abbott Diabetes Care Inc., Alameda, CA (US)

(72) Inventor: Jean-Pierre COLE, Tracy, CA (US)

(21) Appl. No.: 18/903,777

(22) Filed: Oct. 1, 2024

Related U.S. Application Data
(60) Provisional application No. 63/587,295, filed on Oct. 2, 2023.

(51) Int. Cl. A61M 5/315

(52) U.S. Cl. CPC A61M 5/31546

(57) ABSTRACT

A dose monitoring system for an injection pen includes a dose detection assembly and an injection detection assembly. The dose detection assembly includes a housing securable to a body of the injection pen over a dose display window, an optical sensor assembly for determining an amount of movement of a dose indicator surface within the dose display window, and a processor arranged within the housing. The injection detection assembly includes a housing securable to a dose knob of the injection pen, an injection sensor for detecting a force or touch applied to the dose knob, and a transmitter to communicate injection information when the force or touch is applied. The amount of medication injected is determined based on the amount of movement of the dose indicator surface within the dose display window when the force or touch is applied to the dose knob.

### CROSS-REFERENCE TO RELATED APPLICATIONS

This application claims priority to and the benefit of U.S. Provisional Application No. 63/587,295, filed Oct. 2, 2023, which is incorporated herein by reference in its entirety.

### FIELD

Embodiments described herein relate to a dose monitoring system for use with an injection device for automatically determining dose information.

### BACKGROUND

Many individuals require regular administration of medication to manage a disease or illness. For example, individuals with diabetes may require one or more daily insulin injections of one or more medications to help manage their glucose levels, such as long-acting or fast-acting insulin. Medication may be delivered by an injection device, such as an infusion pump, or an injection pen, among others.

Injection pens are commonly used to deliver medication as they are readily available and are relatively inexpensive. The user may select the amount of medication to deliver and self-administer the medication via the injection pen. The injection pen may be disposed of once the medication stored therein is depleted or expired, and the user may obtain and use a new injection pen for further injections of medication.

### SUMMARY OF THE INVENTION

Aspects of the invention are set out in the independent claims and preferred features are set out in the dependent claims. Features of one aspect may be applied to each aspect alone or in combination with other aspects.

Some embodiments described herein relate to a dose monitoring system for an injection pen that includes a dose detection assembly including a housing configured to be secured to a body of the injection pen over a dose display window of the injection pen, an optical sensor assembly configured to determine an amount of movement of a dose indicator surface within the dose display window. The dose monitoring system further includes an injection detection assembly that includes a body configured to be secured to the dose knob of the injection pen, an injection sensor configured to detect a force or touch applied to the dose knob of the injection pen, and a transmitter configured to communicate injection information when the injection sensor detects the force or touch applied to the dose knob. The dose monitoring system includes a processor in communication with one or more of the dose detection assembly or the injection detection assembly that is configured to determine an amount of medication injected based on the amount of movement of the dose indicator surface within the dose display window when the force or touch is applied to the dose knob as detected by the injection sensor.

In any of the various embodiments described herein, the dose detection assembly may include a securement device configured to secure the dose detection assembly to the body of the injection pen or comprises securement means for coupling the dose detection assembly to the body of the injection pen.

In any of the various embodiments described herein, the dose detection assembly may be configured to determine a set dose based on an amount of movement of the dose indicator surface within the dose display window as determined by the optical sensor assembly when no force or touch is detected by the injection sensor. In some embodiments, the dose detection assembly may further include a display configured to display the set dose. In some embodiments, the dose detection assembly may be configured to determine that the set dose was fully injected when the amount of movement within the dose display window when the force or touch is applied to the dose knob is equal to the amount of movement of the dose indicator surface when no force or touch was applied to the dose knob.

In any of the various embodiments described herein, the injection sensor of the injection detection assembly may include a touch sensor.

In any of the various embodiments described herein, the optical sensor assembly may include an optical navigation sensor.

In any of the various embodiments described herein, the optical sensor assembly may include a light source configured to direct light at the dose indicator surface of the injection pen.

In any of the various embodiments described herein, the optical sensor assembly may be configured to detect one or more edges of the dose display window. The optical sensor assembly may comprise means for detecting one or more edges of the dose display window. In some embodiments, the processor may be configured to detect relative movement of the dose detection assembly and the body of the injection pen based on detection of a position of the one or more edges of the dose display window. The assembly may comprise means for detecting the relative movement of the dose detection assembly and the body of the injection pen, optionally based on detection of a position of the one or more edges of the dose display window.

In any of the various embodiments described herein, the transmitter of the injection detection assembly may have a low power mode and a full power mode, wherein the injection detection assembly is configured to transition to the full power mode when the injection sensor is pressed.

Some embodiments described herein relate to a method for detecting dose information by a dose monitoring system coupled to an injection pen, wherein the method includes detecting movement of an indicator surface in a dose display window of an injection pen by an optical sensor assembly of a dose detection assembly of the dose monitoring system, and wherein the dose detection assembly is secured to a body of the injection pen over the dose display window of the injection pen. The method further includes detecting a force or touch applied to an injection sensor of an injection detection assembly of the dose monitoring system, wherein the injection detection assembly is secured to a dose knob of the injection pen, and transmitting injection information from the injection detection assembly to the dose detection assembly when the force or touch is applied to the injection sensor. The method further includes determining, by a processor of the dose monitoring system, an amount of movement of the indicator surface within the dose display window when the force or touch is applied to the dose knob, wherein the amount of movement corresponds to an amount of medication injected.

In any of the various embodiments described herein, the method of detecting dose information may further include transmitting the amount of medication injected to a receiver in wireless communication with the dose monitoring system. In some embodiments, the method may further include displaying dose information on a display of the receiver.

In any of the various embodiments described herein, the method may further include transitioning the injection detection assembly from a low power state to a full power state when force or touch is detected by the injection sensor. In some embodiments, the method may further include transitioning the injection detection assembly from a low power state to a full power state when the injection sensor is pressed according to a predetermined activation pattern.

In any of the various embodiments described herein, the method may further include determining a set dose based on an amount of movement of the dose indicator surface when no force or touch is applied to the injection sensor. In some embodiments, the method may further include displaying the set dose on a display of the dose detection assembly.

In any of the various embodiments described herein, the method may further include detecting relative movement of the dose detection assembly and the body of the injection pen by detecting a change in a position of one or more edges of the dose display window by the optical sensor assembly. In some embodiments, the method may further include correcting the set dose or the injected dose based on the change in the position of the one or more edges of the dose display window.

### BRIEF DESCRIPTION OF THE FIGURES

The accompanying drawings, which are incorporated herein and form a part of the specification, illustrate the present disclosure and, together with the description, further serve to explain the principles thereof and to enable a person skilled in the pertinent art to make and use the same.

FIG. 1 shows an exemplary injection pen for use with a dose monitoring system according to an embodiment.

FIG. 2 shows a view of a dose monitoring system having a dose detection assembly and an injection detection assembly according to an embodiment.

FIG. 3 shows a view of a dose monitoring system of FIG. 2 secured to an exemplary injection pen.

FIG. 4 shows a schematic view of an optical sensor assembly for use in a dose detection assembly according to an embodiment.

FIG. 5 shows a schematic view of the optical sensor assembly of FIG. 4 to detect movement of a dose display window of an injection pen according to an embodiment.

FIG. 6 shows an exemplary method for determining dose information using a dose monitoring system according to an embodiment.

FIG. 7 shows a schematic diagram of components of a dose monitoring system according to an embodiment.

FIG. 8 shows a dose monitoring system having an integrated dose detection assembly and injection detection assembly according to an embodiment.

FIG. 9 shows the dose monitoring system of FIG. 8 secured to an exemplary injection pen.

FIG. 10 shows a schematic diagram of components of a dose monitoring system according to an embodiment.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the claims.

While injection pens are commonly used to administer medication to a user, such injection pens generally have limited functionality and do not assist the user in tracking times or amounts of injections. Rather, the user manually tracks the timing and amount of injections using pen and paper, in a journal or log, or by manual entry on a computer. This can be time-consuming and inconvenient for the user. The user may forget to write down the timing or amount of the medication. The user may also incorrectly record the timing or amounts of medication. The user may for example incompletely administer a set dose, but erroneously record that they fully administered the dose. This can lead to poor compliance with medication administration regimens, and poor or ineffective management of the patient's disease or condition. If accurate records are not kept, it can be difficult for the patient's healthcare professional to determine whether and how to adjust the user's medication regimen.

Studies have found that time in range (“TIR”), which is a metric for tracking a user's glycemic control, is improved in users that administer more daily bolus doses than those that administered fewer daily bolus doses. An average insulin pen user may administer 4-6 daily insulin doses, and some users deliver 7 or more daily insulin doses. Studies have also shown that users who check their glucose levels more frequently, such as by scanning an in vivo analyte sensor to collect analyte data, have improved TIR relative to users who check their glucose levels less frequently. While more frequent monitoring and bolus administration is associated with improved glycemic control, spending more time checking glucose levels and tracking the time and amounts of these insulin doses can be cumbersome for the user, particularly for users who administer a large number of daily doses. Thus, it would be beneficial to automatically track and record the timing and amounts of bolus doses administered, and integrate such dose information with glucose data.

Some injection devices may include dose tracking features integrally formed with the injection device. However, such devices may be prohibitively expensive for many users and may not be available for all medications. Further, as the injection device may be disposable, the user may have to repeatedly purchase injection devices having dose tracking features, which can further add to the cost of the user's treatment. Thus, it would be desirable to provide a dose monitoring system that can be removably coupled to any of various injection pens, such that the dose monitoring system can be reused and can be used with multiple different injection pens.

Some embodiments described herein relate to a dose monitoring system for use with an injection pen that is configured to automatically track and record dose information. The dose monitoring system may wirelessly communicate the collected dose information to a remote server or servers, such as to a cloud computing system, to a receiver, such as a smartphone, tablet, laptop, computer, smartwatch, or other wearable or portable electronic device for storage, analysis, or display to the user. The dose monitoring system helps to ensure that all doses are recorded, and that dose information is recorded accurately.

Some embodiments described herein relate to a dose monitoring system that can be removably coupled to any of various types of injection pens. As a result, the user can continue to use the desired injection pens and can couple the dose monitoring system to the injection pen to collect and determine dose information, and when the injection pen is replaced, the user can continue to use the same dose monitoring system with additional injection pens.

As used herein, “dose information” may refer to any of various information associated with setting and injecting a dose of medication via an injection pen, including but not limited to a set dose amount, a time the dose was set, an injected dose amount, a time of the dose was injected, a time injection is started, a time injection ends, a duration of the injection, a type of medication, a time since a previous injection, or detection of a priming dose, among other information.

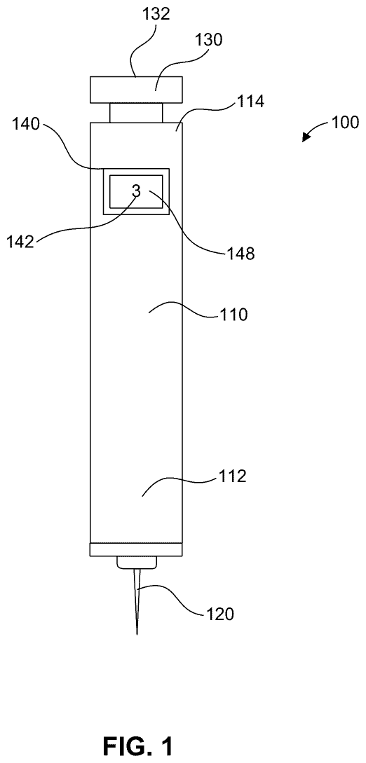

Dose monitoring systems as described herein are configured for use with an injection pen or similar device. While not desiring to be limited to the specific construction of the injection pen, for purposes of illustration an exemplary injection pen 100 for use with dose monitoring systems described herein is shown, for example, in FIG. 1. Injection pen 100 may include a body 110 having a first end 112 opposite a second end 114. Body 110 may have an elongated, tubular shape. A medication container or ampoule containing medication to be administered may be stored within body 110 of injection pen 100 to be administered to the user. An injection tip or needle 120 is arranged at first end 112 of body 110 for piercing the skin of the user and providing a fluid path for administering the medication stored in the medication container. Needle 120 may be removably secured to first end 112 of body 110. For example, body 110 may include threading to receive a threaded needle hub of needle 120.

A dose knob 130 is arranged at second end 114 of body 110 of injection pen 100 for setting a dose of medication to be administered. In operation, a user may set a dose by rotating dose knob 130 in a clockwise or counter-clockwise direction to increase or decrease the set dose. Dose knob 130 may rotate and/or may move inward and outward from body of injection pen 100 during dose setting depending on the specific injection pen.

Injection pen 100 may include a dose display window 140 for displaying the set dose. A dose indicator surface 148 may be arranged within dose display window 140 and includes a series of markings 142 (e.g., numerals, lines, hashes) on dose indicator surface 148 to indicate the currently set dose amount. Dose indicator surface 148 may rotate along with dose knob 130 so that dose indicator surface 148 moves and increments the set dose as dose knob 130 is rotated. After the desired dose is set, an end surface 132 of dose knob 130 may be pressed in a longitudinal direction of injection pen 100, i.e., in a direction toward first end 112 of injection pen 100, to administer the set dose.

Injection pen 100 further includes internal components for driving a flow of medication out of needle 120 of injection pen 100, such as a piston or plunger that moves through a medication container or ampoule, and a mechanical arrangement for transmitting force applied to the dose knob 130 to the plunger to drive the medication from the medication container through the needle 120. As the dose knob 130 is pressed, the dose indicator surface 148 may return from the set dose position to a zero dose position for the next administration of medication.

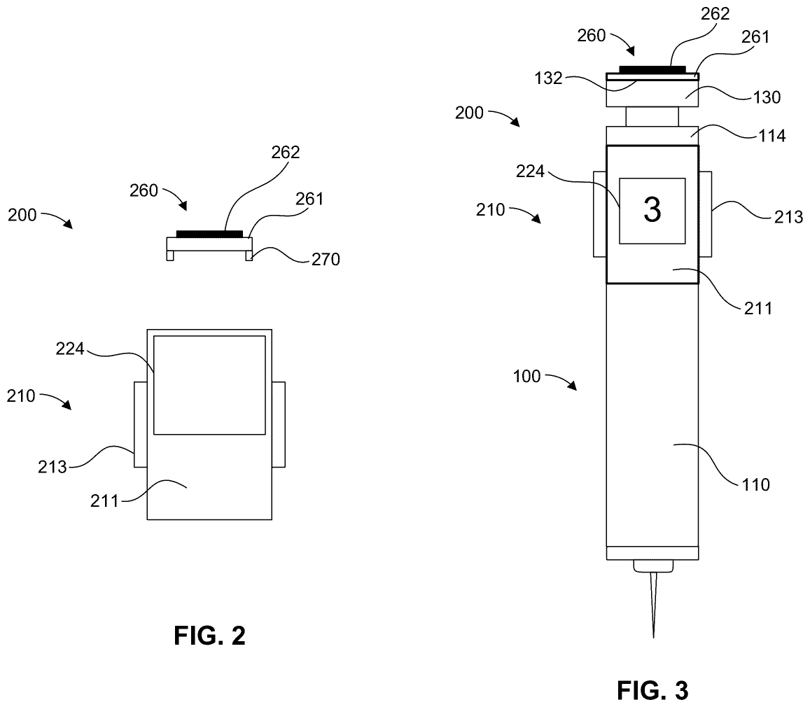

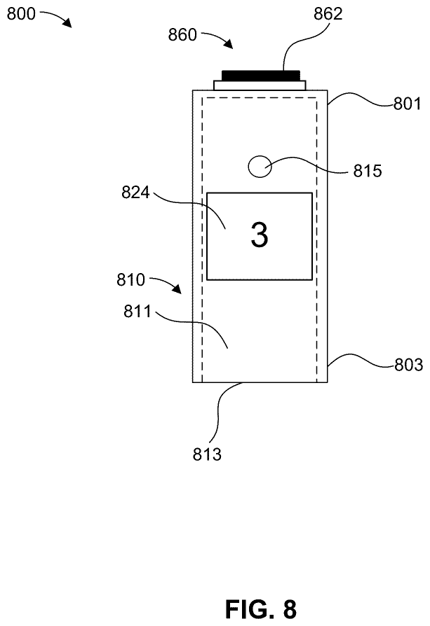

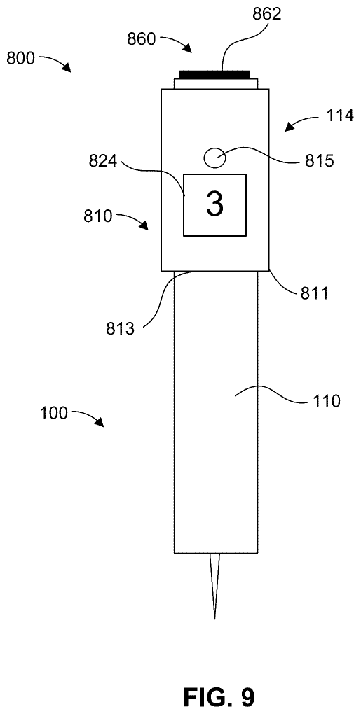

Some embodiments described herein relate to a dose monitoring system 200 that includes a dose detection assembly 210 and an injection detection assembly 260, as shown for example in FIGS. 2 and 3. Dose detection assembly 210 and injection detection assembly 260 may be separate components and may be in wired or wireless communication with one another, such as by a Bluetooth communication protocol or other short range wireless communication protocols. In some embodiments, however, dose detection assembly 210 and injection detection assembly 260 may be combined or integrally formed as a one-piece assembly or single device (see, e.g., FIGS. 8-9). Dose detection assembly 210 may be configured to be removably secured to a body 110 of injection pen 100 and is configured to detect movement of dose indicator surface 148 within dose display window 140 to aid in determining set dose and injected dose amounts. Injection detection assembly 260 may be configured to be secured to dose knob 130 of injection pen 100 and is configured to detect a touch or force applied to dose knob 130, such as application of force to inject medication. Injection detection assembly 260 may communicate injection information to dose detection assembly 210 for use in determining dose information. In some embodiments, injection information may be communicated alternatively or additionally to a remote server or to a receiver for use in determining dose information.

In some embodiments, dose detection assembly 210 may include a housing 211 configured to be secured to an injection pen 100. Specifically, housing 211 may be configured to be secured to a body 110 of injection pen 100 toward second end 114 of body 110. Housing 211 may include one or more securement devices 213 configured to removably secure housing 211 to body 110 of injection pen 100. Securement device 213 may include one or more arms configured to wrap around at least a portion of body 110 of injection pen 100, such as circumferentially around body 110 of injection pen 100. In some embodiments, securement device 213 may include a bore formed by a portion of housing 211 and configured to receive body 110 of injection pen 100 therethrough. In some embodiments, securement device 213 may include one or more clips for at least partially encircling a portion of body 110. Securement device 213 may be configured to adjust to fit securely to any of various types of injection pens having different cross-sectional shapes (oval, circle, square, etc.) and different dimensions. Dose detection assembly 210 is configured to be secured to injection pen 100 in a fixed position and to prevent relative movement between dose detection assembly 210 and injection pen 100.

Dose detection assembly 210 is configured to be positioned over dose display window 140 of injection pen 100. Dose detection assembly 210 includes a sensor assembly 230 configured to detect movement of dose indicator surface 148 within dose display window 140, as discussed in further detail herein. Dose detection assembly 210 may further include a display 224 configured to display a set dose, among other information (e.g., dose information). As dose detection assembly 210 covers dose display window 140 of injection pen 100, display 224 of dose detection assembly 210 may display the currently set dose so that the user may view the current dose setting. As the user dials the dose, such as by rotating dose knob 130, dose detection assembly 210 may determine the set dose and the set dose amount may be shown on display 224 to allow the user to view the set dose. In some embodiments, the set dose may be shown on a receiver in communication with the dose monitoring system 200.

In addition to dose detection assembly 210, dose monitoring system 200 includes injection detection assembly 260. Injection detection assembly 260 may include a body 261 configured to be mounted to dose knob 130 of injection pen 100. Body 261 may have a cross sectional shape similar to that of dose knob 130, and may have a circular cross sectional shape. Body 261 may be removably secured to end surface 132 of dose knob 130, such as by friction fit, interference fit, snap fit, or the like. In some embodiments, injection detection assembly 260 may be configured to mate with dose knob 130 such by mating or engaging portions, such as by a threaded connection, or by engagement of a ridge or groove of body 261 with a portion of dose knob 130, among others removable fastening arrangements. In some embodiments, body 261 may include a lip 270 configured to encircle a portion of dose knob 130 to facilitate securement of injection detection assembly 260 to dose knob 130. Lip 270 may be configured to be positioned circumferentially around dose knob 130. In some embodiments, body 261 may include a pressure-sensitive adhesive for securing injection detection assembly 260 to end surface 132 of dose knob 130.

Injection detection assembly 260 includes an injection sensor 262. Injection sensor 262 may include a touch sensor, such as a resistive or capacitive touch sensor, or a force sensor, such as a force-sensing resistor. Injection sensor 262 may detect application of touch or force applied to injection detection assembly 260 and thus to dose knob 130 in order to determine that an injection is taking place. Injection detection assembly 260 may be in communication with dose detection assembly 210 and may transmit injection information, such as an indication that injection sensor 262 has been pressed or released, the time injection sensor 262 was first activated or pressed, the duration for which injection sensor 262 is pressed, the time at which injection sensor 262 is released (no longer pressed), or the amount of force applied to injection sensor 262, among other information. In some embodiments, the injection information may alternatively or additionally be transmitted to a remote server or to a receiver.

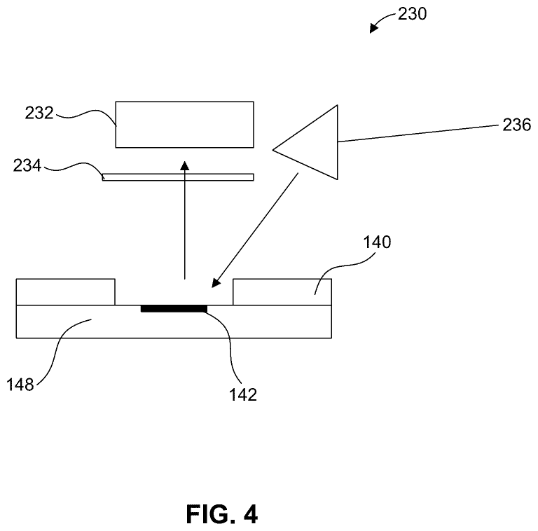

In order to determine a set dose amount, dose detection assembly 210 includes a sensor assembly 230 configured to detect movement of dose indicator surface 148 within dose display window 140, as shown for example in FIG. 4 as discussed in further detail below.

A sensor assembly as described herein may include an optical sensor, such as an optical navigation sensor, as discussed in further detail below. As the dose is set or dialed, dose indicator surface 148 of injection pen 100 moves to show the markings 142 indicative of the set dose amount in dose display window 140. Sensor assembly 230 of dose detection assembly 210 determines the amount of movement of dose indicator surface 148, and dose detection assembly 210 may correlate the amount of movement with the set dose amount. The amount of movement may be the distance measured along the dose indicator surface from an initial position of dose indicator surface (prior to dose setting) and a second position corresponding to the set dose. The set dose may be output on a display of dose detection assembly 210. Sensor assembly 230 may detect markings 142 on dose indicator surface 148, such as numerals or hash lines. Sensor assembly 230 may capture a plurality of images as the dose indicator surface 148 moves. In some embodiments, sensor assembly 230 may take one or more images per second, and may take 10 or more images per second, 50 or more images per second, or 100 or more images per second. Sensor assembly 230 can detect the direction of movement of dose indicator surface 148, such as movement in a first direction (increasing the set dose) or movement in a second opposing direction (decreasing the set dose). This allows dose detection assembly 210 to account for situations in which the user dials a dose and then corrects the dialed dose, such as to decrease the set dose to a smaller amount.

In some embodiments, sensor assembly 230 may include an optical navigation sensor as shown for example in FIG. 4. Optical navigation sensor as used herein may be similar to an optical navigation sensor of the type used in an optical computer mouse. In such embodiments, sensor assembly 230 may include a light source 236 configured to illuminate dose indicator surface 148 in dose display window 140. Light source 236 may be a laser, an LED light, or an infrared light source, among others. The light produced by light source 236 is reflected off of dose indicator surface 148 toward sensor 232. A lens 234 may be used to focus reflected light toward sensor 232. Sensor assembly 230 may detect movement of dose indicator surface 148 based on differences on the surface of dose indicator surface 148, including for example the markings 142 on the surface (e.g., 1, 2, 3, etc.), hash lines, among other features. Dose detection assembly 210 receives information from sensor assembly 230, such as the amount and direction of movement, and correlates the amount and direction of movement with a set or injected dose amount.

In some embodiments, dose monitoring system 200 may require an initial calibration to determine the relationship of the amount of movement of dose indicator surface to a set dose amount. In some embodiments, dose monitoring system 200 may prompt a user to set the dose to a predetermined amount. For example, dose monitoring system 200 may prompt the user to set the dose to 1 unit. The user may then set the dose to 1 unit by rotating the dose knob of the pen and then confirm that the dose has been set to 1 unit. System 200 can then determine the amount of movement of the dose indicator surface that corresponds to the dose setting of 1 unit. This calibration information can then be used to determine the dose setting corresponding to a given amount of movement of dose indicator surface 148. In some examples, dose monitoring system 200 may require user calibration at predetermined time intervals (e.g., once a week, once a month, or any other predetermined interval). In some examples, dose monitoring system 200 may allow a user to initiate a calibration at any time. Alternatively, dose monitoring system 200 may prompt the user to enter the type of pen, such as the specific brand or model of pen, and system 200 may retrieve calibration information for that particular pen, whether from internal memory or from a remote computing device or server (e.g., cloud).

Dose detection assembly 210 may be fixed securely to injection pen 100 such that there is no relative movement between dose detection assembly 210 and injection pen 100. Error may be introduced in the determination of the set dose in the event that housing 211 of dose detection assembly 210 moves relative to injection pen 100 during dose setting or injection, as the relative movement of housing 211 and injection pen 100 may impact the amount of movement detected by sensor assembly 230. However, to the extent that some relative movement may occur, in some embodiments, system 200 may be configured to detect or compensate for the relative movement.

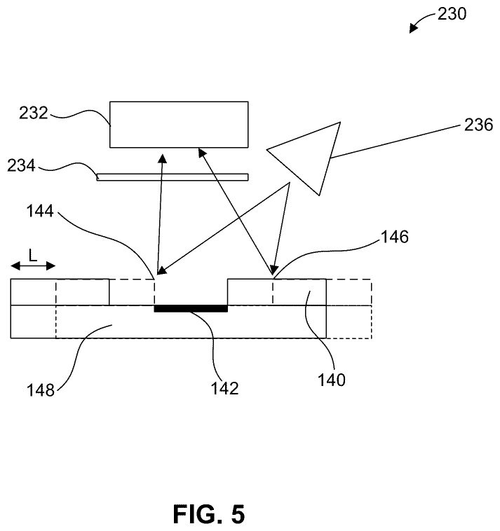

As shown for example in FIG. 5, dose display window 140 may move relative to dose detection assembly 210 such as due to unintended rotation of dose detection assembly 210 on injection pen 100. Dose display window 140 may move from first position (shown in solid lines) with respect to sensor assembly 230 to a second position (shown in dotted lines), such as by distance L. Dose detection assembly 210 may be configured to detect a location of dose display window 140 of injection pen 100, such as by locating one or more edges 144, 146 of dose display window 140. If dose detection assembly 210 moves relative to injection pen 100, a location of dose display window 140 relative to sensor assembly 230 will change. Sensor assembly 230 may determine the amount of movement L of the location of dose display window 140.

If movement of dose display window 140 is detected, dose detection assembly 210 may transmit a signal to a receiver to output a notification that the set dose determined by dose monitoring system 200 may be inaccurate. Dose detection assembly 210 may only transmit the signal to the receiver if the amount of movement is greater than or equal to a predetermined minimum amount of movement. In some embodiments, dose detection assembly 210, or receiver, may determine a corrected set dose by accounting for the movement of the dose display window 140 when determining the set or injected dose. For example, if movement of dose indicator surface 148 in a first amount is determined, that amount may be offset by the amount of movement L of dose display window 140. In this way, dose monitoring system 200 may compensate for relative movement between dose detection assembly 210 and injection pen 100.

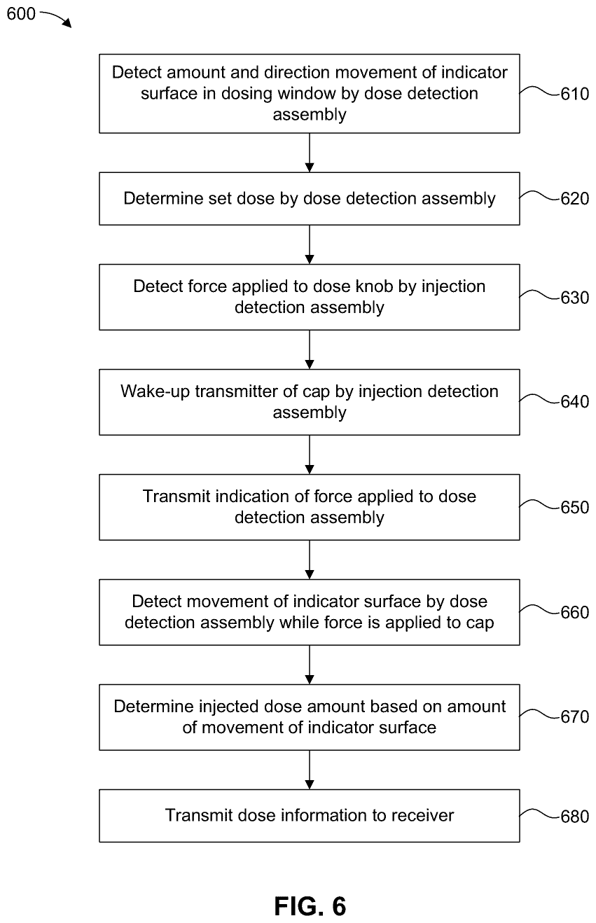

An exemplary method 600 of using a dose monitoring system as described herein is shown for example in FIG. 6. As a user dials dose of medication by rotating dose knob of injection pen, dose detection assembly detects an amount and direction of movement of dose indicator surface in dose display window of injection pen when injection sensor is not pressed 610. Dose detection assembly determines the set dose based on the detected amount of movement 620. The determined set dose may be displayed on a display of the dose detection assembly. Injection detection assembly may detect a force or touch applied to dose knob of injection device 630. In some embodiments, application of touch or force to dose knob may wake-up transmitter of injection detection assembly 640 (e.g., transition from a low power state to a full power state). Transmitter of dose injection assembly may transmit injection information to the dose detection assembly 650. Alternatively or additionally, injection information can be transmitted to a remote server or to a receiver. While touch is detected (or while force is applied) to injection detection assembly, dose detection assembly may detect movement of the dose indicator surface in dose display window of injection pen 660. Dose monitoring system may determine the injected dose amount based on the amount of movement of the dose indicator surface while touch or force is applied to injection detection assembly 670. Dose monitoring system may transmit dose information to a receiver 680. In some embodiments, collected data, such as injection information, and data relating to movement of the dose indicator surface may be transmitted to a remote device, such as to a cloud computing system or to the receiver, such that the cloud computing system or receiver may determine the injected dose amount among other dose information. Dose information may be determined by any of the dose monitoring system, a remote server, a receiver in communication with the dose monitoring system, or by a combination thereof.

In some embodiments, transmitter of dose injection assembly may remain in a full power state so that the step of waking-up the transmitter 640 may be omitted. In some embodiments, dose injection assembly may wake-up prior to use of the injection pen, such as by touching or applying a force to the injection sensor in accordance with an activation pattern prior to dialing a dose as discussed in further detail herein.

Dose monitoring system may determine that a set dose is fully injected if the amount of movement when the injection sensor is not pressed is equal to the amount of movement of the dose indicator surface when the injection sensor is pressed. Dose monitoring system may determine that a set dose is partially injected if the amount of movement when the injection sensor is pressed is less than the amount of movement detected for the set dose (when the injection sensor was not pressed). The dose monitoring system may separately store and transmit to the receiver both the set dose and the amount of medication actually injected (i.e., a partial dose).

In some embodiments, during setting a dose, dose detection assembly may consider movement of the dose indicator surface in either direction (clockwise or counterclockwise) which may correspond to increasing or decreasing the selected dose. In some embodiments, however, during injection, dose detection assembly may only consider movement of dose indicator surface in one direction (i.e., the set dose returning to or toward a zero dose setting), as movement in the opposite direction may be the result of error or improper operation of the injection pen.

In some embodiments, dose monitoring system 200 may be configured to detect a priming dose. Prior to administering a dose of medication, a user may prime the pen by selecting and ejecting a small amount of medication from the injection pen (e.g., 2 units). Dose monitoring system 200 may detect a priming dose by detecting ejection of a first dose of medication of an amount less than a predetermined threshold and subsequent ejection of a dose of medication that is greater than the predetermined threshold. Dose monitoring system 200 may determine that the first dose is a priming dose. Dose monitoring system 200 may further determine a time of the first dose and a time of the second dose, and a time between the first and second doses. Dose monitoring system 200 may determine that the first dose of the two doses is a priming dose if the time between the first and second doses is less than a predetermined period of time (e.g., 90 seconds, 60 seconds, or 30 seconds, among other periods of time).

In some embodiments, a machine learning model may be used to determine dose information. A machine learning model may receive as an input dose information, including a dose history, set dose amounts, injected dose amounts, dose administration times, or duration injection sensor is pressed, among other dose information, and combinations thereof. Machine learning model may be used to classify a dose of medication as a priming dose or as an actual dose, or as a meal dose or correction dose, or as a breakfast dose, lunch dose, or dinner dose, among others.

The model can be provided with dose information from dose monitoring system. Machine learning models can include, by way of example and not limitation, models trained using or encompassing decision tree analysis, gradient boosting, adaptive boosting, artificial neural networks or variants thereof, linear discriminant analysis, nearest neighbor analysis, support vector machines, supervised or unsupervised classification, and others. The models can also include algorithmic or rules-based models in addition to machine learned models. Machine learning involves computers discovering how they can perform tasks without being explicitly programmed to do so. Machine learning includes, but is not limited to, artificial intelligence, deep learning, fuzzy learning, supervised learning, and unsupervised learning, etc.

Machine learning algorithms may build an initial prediction model based on sample data, known as “training data”, in order to make predictions or decisions without being explicitly programmed to do so. This sample data may include injection data from the patient or from a population of patients. For supervised learning, the computer is presented with example inputs and their desired outputs and the goal is to learn a general rule that maps inputs to outputs. In another example, for unsupervised learning, no labels are given to the learning algorithm, leaving it on its own to find structure in its input. Unsupervised learning can be the ultimate goal such as discovering trends or patterns in data, or a means towards another goal, such as improved accuracy of future predictions.

A machine-learning engine may use various classifiers to map concepts or data to capture relationships between concepts (e.g., time, time of day, dose amount, dose times, and other dose information) and an accuracy of prior predicted patient outcomes. The classifier (discriminator) is trained to distinguish (recognize) in variations of data. In some aspects, machine learning models are trained on a remote machine learning platform using a history of dose information from the patient, or from a population of patients. In one embodiment, prediction models are continuously updated as new patient information is received.

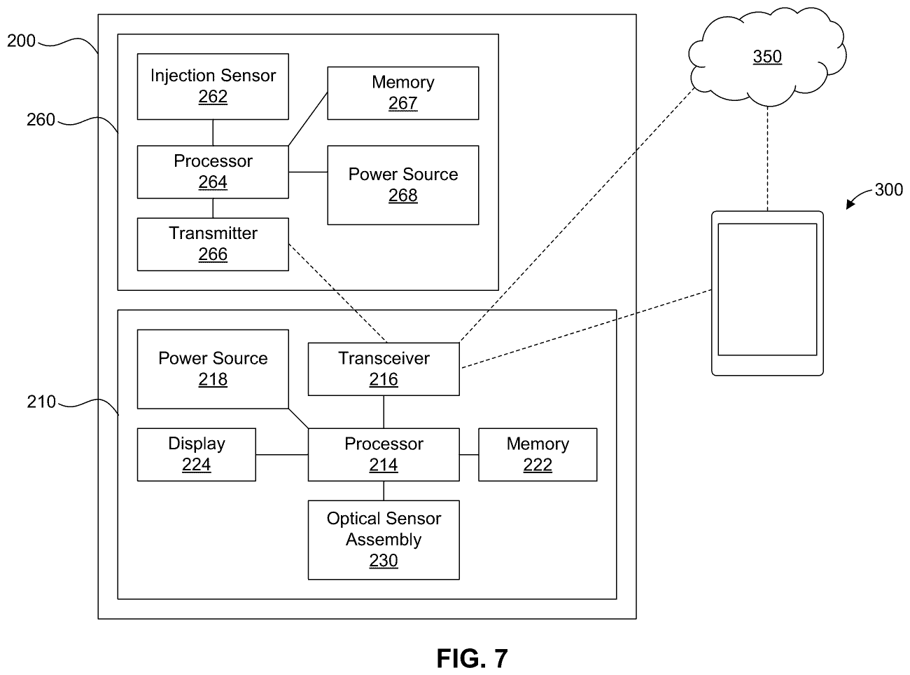

A schematic diagram of components of a dose monitoring system according to an embodiment is shown in FIG. 7. Dose detection assembly 210 may include one or more processors 214 and a memory 222 coupled to the one or more processors 214. Memory 222 may store programming and instructions for determining dose information as discussed herein. Memory 222 may further store dose information collected and determined by dose monitoring system 200. A display 224 of dose monitoring assembly may be coupled to processor 214 for displaying set dose information, among other information to the user. Dose detection assembly 210 may include a power source 218 for powering the components thereof. Power source 218 may include one or more batteries. Power source 218 may be rechargeable, and in some embodiments, housing of dose detection assembly 210 may include a port for connection to a cable for charging power source 218. In some embodiments, power source 218 may be configured to be wirelessly charged, such as by inductive charging. Dose detection assembly 210 further includes a sensor assembly 230 for detecting movement of a dose indicator surface of the injection pen, such as an optical sensor assembly as discussed herein. Dose detection assembly 210 may include a transceiver 216 for communicating with injection detection assembly 260, a receiver 300, and/or a remote server(s) 350.

Injection detection assembly 260 may include an injection sensor 262 is configured to detect a force or touch applied to injection detection assembly 260 as described herein. Injection detection assembly 260 may include a transmitter 266 to transmit injection information to dose detection assembly 210 and/or receiver 300. In some embodiments, injection detection assembly 260 may further be configured to receive information from dose detection assembly 210, and thus transmitter 266 may be a transceiver. Injection detection assembly 260 may include a power source 268 configured to provide energy to its components. Power source 268 may include one or more batteries. Power source 268 may be rechargeable, and in some embodiments, body of injection detection assembly 260 may include a port for connection to a cable for charging power source 268. In some embodiments, power source 268 may be configured to be wirelessly charged, such as by inductive charging. Injection detection assembly 260 may include one or more processors 264 coupled to injection sensor 262, transmitter 266, and power source 268. Processor 264 may be coupled to a memory 267 storing instructions or programming for operation of injection detection assembly 260 and for determining dose information as described herein. Memory 267 may store collected information, such as information regarding detection of a touch or force applied to injection sensor 262, a time injection sensor is touched, a time injection sensor is released, and a duration injection sensor is pressed, a force applied to injection sensor, among other information.

Injection detection assembly 260 may be in a low-power state or sleep state when not in use in order to conserve energy. When injection sensor 262 is pressed, processor 264 may be configured to wake-up injection detection assembly 260 and transition to a full-power state in order to transmit injection information to dose detection assembly 210 or to receiver 300. In some embodiments, injection detection assembly 260 may remain in the sleep state until the injection sensor is pressed according to a predetermined activation pattern. For example, the user may be required to press the injection sensor in a predetermined manner, such as to double-tap injection sensor 262 (or triple-tap) or to press and hold injection sensor 262 for a predetermined period of time (e.g., 3 seconds, 5 seconds, or 10 seconds) prior to dialing a dose, in order to wake-up the injection detection assembly 260. This may help to prevent accidental wake-up of the injection detection assembly 260, such as if injection pen used with dose monitoring system is stored in a bag or pocket, or if the injection pen is dropped.

Dose monitoring system 200 may communicate with a receiver 300, such as to transmit dose information. In some embodiments, dose detection assembly 210, injection detection assembly 260, or both, may be configured to communicate with the receiver 300. Communication may be through a wireless communication protocol, such as a Bluetooth communication protocol or other short range wireless communication protocols. Various types of dose information as described herein may be determined by one or more of dose detection assembly 210, injection detection assembly 260, receiver 300, or by a remote server in communication with dose monitoring system 200. For example, injection detection assembly 260 may detect a time injection sensor 262 is pressed and a time injection sensor 262 is released, and may determine the duration of the injection. Alternatively or additionally, injection detection assembly 260 may transmit the time the injection sensor 262 is pressed and the time the injection sensor 262 is released to dose detection assembly 210, and dose detection assembly 210 may determine the duration of the injection. In another example, dose detection assembly 210 may detect the amount of movement of dose indicator surface when injection sensor is pressed, and may determine the injected dose amount. Alternatively, dose detection assembly 210 may transmit to a receiver 300 the amount of movement of the dose indicator surface when the injection sensor is pressed such that receiver 300 determines the injected dose amount.

In some embodiments, receiver 300 may include a mobile electronic device, such as a dedicated receiver, a smartphone, a tablet, a smartwatch, or a laptop, among other portable or wearable electronic devices. Receiver 300 may include a user input, such as one or more buttons, touch screen, or the like, and a user interface including a display for displaying information, such as dose information, to the user. Receiver 300 may include one or more processors, memory coupled to the one or more processors and storing instructions for receiving and processing data from a dose monitoring system 200. Receiver 300 may include a power source, such as one or more disposable or rechargeable batteries. Receiver 300 may include communication circuitry, such as a transceiver for wirelessly communicating with dose monitoring system by a wireless communication protocol, such as a Bluetooth communication protocol.

Receiver 300 may be configured to store and display dose information or other data received from dose monitoring system 200. After a dose is injected, receiver 300 may display dose information such as the time of the dose and the amount of the dose injected. The user may confirm the dose information is accurate, and may be prompted to accept or edit the displayed dose information. The user may input additional information, such as the type of medication, or notes associated with the medication dose, which can be saved in an injection log.

Receiver 300 may be configured to store and display an injection log that includes dose information, such as time of injections, amount of injections, type of medications injected, and user notes, among other information. Receiver 300 may determine and display additional data, metrics, or statistics based on the received dose information. In some embodiments, receiver 300 may be configured to calculate the total amount of medication administered over a period of time. Receiver 300 may log priming doses separately from injected doses. Receiver 300 may track missed doses and partially delivered doses, and may provide metrics relating to patient compliance with a treatment protocol. In some embodiments, receiver 300 may track an amount of medication remaining in an injection pen 100 based on a known amount of medication in pen prior to use and the user's injection history. Receiver 300 may provide an alert notifying the user that the pen is low on medication and may need to be replaced soon.

Receiver 300 and dose monitoring system, e.g., dose monitoring system 200, may be paired in order to enable data to be communicated between receiver 300 and dose monitoring system 200. Receiver 300 may be configured to prompt the user to enter a code associated with dose monitoring system 200. Code of dose monitoring system 200 may be manually entered into receiver 300 as part of the process of pairing the receiver 300 and dose monitoring system 200 for communication. Alternatively, receiver 300 may include a camera or optical sensor to read a barcode or QR code on dose monitoring system 200 or packaging thereof. This helps to improve data security and prevent sensitive patient data from being intercepted by third party devices. The dose monitoring system 200 may encrypt the data prior to transmission and the receiver 300 may be configured to decrypt the data for storage and/or display. Receiver 300 may be password protected and may require the user to enter a password in order to view dose information provided by dose monitoring system 200.

Receiver 300 and/or dose monitoring system 200 may further communicate with a remote server or servers (e.g., a cloud) 350. Remote server may determine dose information, store dose information, and may receive and store the user's dose information and metrics from receiver or dose monitoring system. In some embodiments, dose monitoring system 200 may communicate directly with remote server 350. In other embodiments, dose monitoring system 200 may communicate with receiver 300 which in turn communicates with remote server 350.

In some embodiments, dose monitoring system 200 or receiver 300 may be configured to provide a reminder to deliver an injection. In some embodiments, receiver 300 may display a time since the last injection. The time may be determined by receiver 300 or by dose monitoring system 200. In some embodiments, receiver 300 may be configured to output an alert or notification to provide a reminder if the time since the last injection exceeds a predetermined amount of time. In some embodiments, a user may input an injection schedule, such as scheduled times to administer injections, and dose monitoring system 200 or receiver 300 may provide an alert, such as one or more of an audible, vibratory, or visual alert to notify the user of one or more of an upcoming injection (e.g., 15 minutes before the injection time), at the time of the injection, or after a scheduled injection if the injection was not administered (e.g., a dose was scheduled to be administered but no dose was recorded). In some embodiments, dose monitoring system 200 may automatically determine an injection schedule based on the user's injection history. Dose monitoring system 200 may determine a pattern of injection times after being used for one or more days and may determine injection times based on past injection times. Dose monitoring system 200 may provide alerts based on the detected injection schedule.

In some embodiments, a machine learning model may be used to determine an injection schedule based on the dose information. The machine learning model may receive as an input the user's dose information, such as dose times, dose amounts, and determine an injection schedule of the user based on analysis of the dose information over time. Machine learning model may further classify the doses as a meal dose, correction dose, or priming dose based on the dose information. Alerts may be provided based on the injection schedule, such as an alert to preemptively notify the user of an upcoming dose, an alert to take a dose at that time, or a missed dose alert to notify the user that he or she may have missed a dose. In summary, a dose monitoring system for an injection pen includes a dose detection assembly and an injection detection assembly. The dose detection assembly includes a housing securable to a body of the injection pen over a dose display window, an optical sensor assembly for determining an amount of movement of a dose indicator surface within the dose display window, and a processor arranged within the housing. The injection detection assembly includes a housing securable to a dose knob of the injection pen, an injection sensor for detecting a force or touch applied to the dose knob, and a transmitter to communicate injection information to the dose detection assembly when the force or touch is applied. The processor of the dose detection assembly determines an amount of medication injected based on the amount of movement of the dose indicator surface within the dose display window when the force or touch is applied to the dose knob among other dose information. In some embodiments, injection information may be transmitted by the injection detection assembly or by the dose detection assembly to a cloud or to a receiver, and data relating to the movement of the dose indicator surface may be transmitted to the cloud or to the receiver, such that the cloud or the receiver may determine the injected dose amount among other dose information.

Some embodiments described herein relate to a dose monitoring system 800 that includes an integrated dose detection assembly 810 and an injection detection assembly 860, as shown for example in FIGS. 8 and 9. Dose monitoring system 800 may have the same or similar features and functionality as described above with respect to dose monitoring system 200. Dose monitoring system 800 may include a housing 811 configured to be secured to an injection pen 100. Housing 811 may include a first end 801 opposite a second end 803. Specifically, housing 811 may be configured to be secured to a body 110 of injection pen 100 toward second end 114 of body 110. Housing 811 may include one or more securement devices 813, as described herein, configured to removably secure housing 811 to body 110 of injection pen 100. Securement device 813 may include a bore extending from second end 803 of housing 811 into housing 811 toward first end 801. Housing 811 may include one or more locators configured to facilitate alignment of a sensor assembly over dose display window 140 of injection pen 100. Locators may include a protrusion configured to engage a portion of dose display window 140.

System 800 may include a display 824 may show dose information, such as a set dose amount, injected dose amount, among other information. Display 824 may be arranged on housing 811. System 800 may further include one or more input devices 815 to receive a user input. Input devices 815 may be arranged on housing 811. Input devices 815 may include one or more of a button, wheel, dial, switch, touch sensor, among others.

System 800 may include an injection detection assembly 860. Injection detection assembly 860 may be coupled to or integrally formed with housing 811. Injection detection assembly 860 may include a portion configured to be mounted to dose knob of injection pen 100. Injection detection assembly 860 may include an injection sensor 862 as described herein for detecting a force or touch applied to injection detection assembly 860, and thus to the dose knob of injection pen.

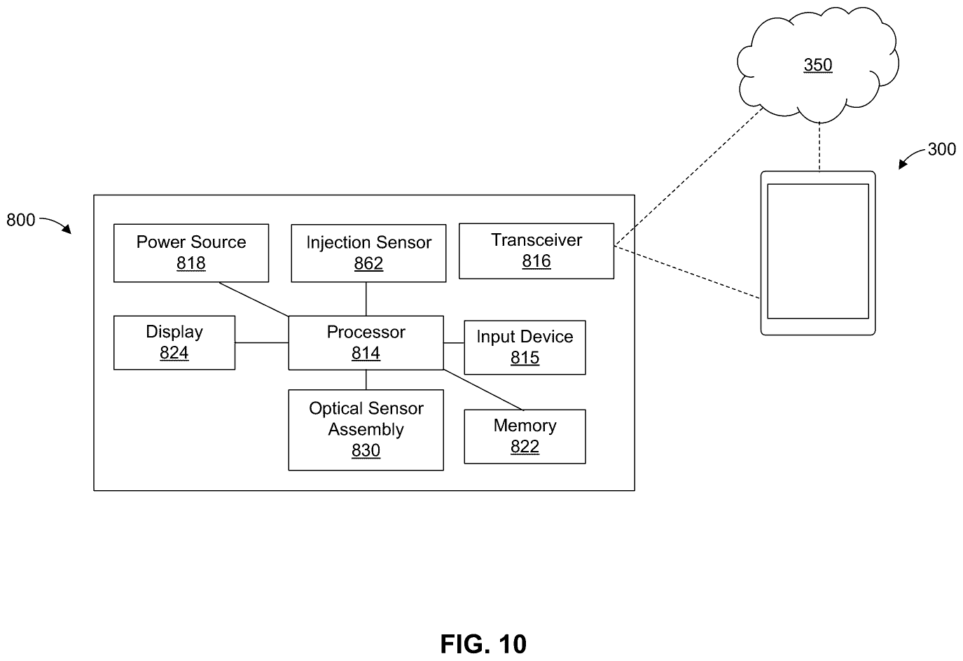

In some embodiments, a schematic diagram of components of a dose monitoring system 800 is shown in FIG. 10. Dose monitoring system 800 includes a dose monitoring assembly and injection detection assembly (as in FIGS. 8-9) that are combined into a single device. Dose monitoring system 800 may include a battery, transceiver and/or processor that are in communication with optical sensor assembly 830 and injection sensor 862. This may reduce the number of components of system relative to dose monitoring system 200 which includes a dose monitoring assembly and injection detection assembly that may have separate batteries, processors, and transceivers.

Dose monitoring system 800 may include one or more processors 814 and a memory 822 coupled to the one or more processors 814. Memory 822 may store programming and instructions for determining dose information as discussed herein. Memory 822 may further store dose information collected and determined by dose monitoring system 800 to be transferred to another device. A display 824 of dose monitoring system 800 may be coupled to processor 814 for displaying information, such as set dose amount, among other information to the user. Dose monitoring system 800 may include a power source 818 for powering the components thereof. Power source 818 may include one or more batteries. Power source 818 may be rechargeable, and in some embodiments, housing 811 of dose monitoring system 800 may include a port for connection to a cable for charging power source 818. In some embodiments, power source 818 may be configured to be wirelessly charged, such as by inductive charging. System 800 includes an injection sensor 862 for detecting a force or touch applied to a dose knob of injection pen 100. Dose monitoring system 800 further includes a sensor assembly 830 for detecting movement of a dose indicator surface of the injection pen, such as an optical sensor assembly as discussed herein. Dose monitoring system 800 may include a transceiver 816 for communicating with a receiver 300, and/or a remote server(s) 350 (e.g., cloud).

The Summary and Abstract sections may set forth one or more but not all exemplary embodiments of the present invention(s) as contemplated by the inventors, and thus, are not intended to limit the present invention(s) and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention(s) that others can, by applying knowledge within the skill of the art, readily modify and/or adapt for various applications such specific embodiments, without undue experimentation, and without departing from the general concept of the present invention(s). Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance herein.

Exemplary embodiments are set out in the following numbered clauses:

1. A dose monitoring system for an injection pen, the dose monitoring system comprising:
a dose detection assembly, comprising:
   a housing configured to be secured to a body of the injection pen over a dose display window of the injection pen,
   an optical sensor assembly configured to determine an amount of movement of a dose indicator surface within the dose display window, and
   a processor arranged within the housing; and
an injection detection assembly, comprising:
   a housing configured to be secured to a dose knob of the injection pen,
   an injection sensor configured to detect a force applied to the dose knob of the injection pen, and
   a transmitter configured to communicate injection information when the injection sensor detects the force applied to the dose knob,
   wherein the processor is configured to determine an amount of medication injected based on the amount of movement of the dose indicator surface within the dose display window when the force is applied to the dose knob.

2. A dose monitoring system for an injection pen, the dose monitoring system comprising:
a dose detection assembly, comprising:
   a housing configured to be secured to a body of the injection pen over a dose display window of the injection pen, and
   an optical sensor assembly configured to determine an amount of movement of a dose indicator surface within the dose display window;
an injection detection assembly, comprising:
a body configured to be secured to a dose knob of the injection pen,
   an injection sensor configured to detect a force or touch applied to the dose knob of the injection pen, and
   a transmitter configured to communicate injection information when the injection sensor detects the force or touch applied to the dose knob; and
   a processor in communication with the optical sensor and the injection sensor that is configured to determine an amount of medication injected based on the amount of movement of the dose indicator surface within the dose display window when the force or touch is applied to the dose knob as detected by the injection sensor.

3. The system of clause 1 or 2, wherein the dose detection assembly comprises a securement device configured to secure the dose detection assembly to the body of the injection pen.

4. The system of clause 1, 2 or 3, wherein the dose detection assembly is configured to determine a set dose based on an amount of movement of the dose indicator surface within the dose display window as determined by the optical sensor assembly when no force or touch is detected by the injection sensor.

5. The system of any of clauses 1 to 4, wherein the dose detection assembly further comprises a display configured to display one or more of the set dose or the amount of medication injected.

6. The system of clause 4 or 5, wherein the dose detection assembly is configured to determine that the set dose was fully injected when the amount of movement within the dose display window when the force or touch is applied to the dose knob is equal to the amount of movement of the dose indicator surface when no force or touch was applied to the dose knob.

7. The system of any of clauses 1 to 6, wherein the injection sensor of the injection detection assembly comprises a touch sensor.

8. The system of any of clauses 1 to 7, wherein the optical sensor assembly comprises an optical navigation sensor.

9. The system of any of clauses 1 to 8, wherein the optical sensor assembly comprises a light source configured to direct light at the dose indicator surface of the injection pen.

10. The system of any of clauses 1 to 9, wherein the optical sensor assembly is configured to detect one or more edges of the dose display window.

11. The system of clause 10, wherein the processor is configured to detect relative movement of the dose detection assembly and the body of the injection pen based on detection of a position of the one or more edges of the dose display window.

12. The system of any of clauses 1 to 11, wherein the transmitter of the injection detection assembly comprises a low power mode and a full power mode, wherein the injection detection assembly is configured to transition to the full power mode when the injection sensor is pressed.

13. A method for detecting dose information by a dose monitoring system coupled to an injection pen, the method comprising:
detecting movement of an indicator surface in a dose display window of an injection pen by an optical sensor assembly of a dose detection assembly of the dose monitoring system, wherein the dose detection assembly is secured to a body of the injection pen over the dose display window of the injection pen;
detecting a force or touch applied to an injection sensor of an injection detection assembly of the dose monitoring system, wherein the injection detection assembly is secured to a dose knob of the injection pen;
transmitting injection information from the injection detection assembly to the dose detection assembly when the force or touch is applied to the injection sensor; and
determining, by a processor of the dose monitoring system or dose detection assembly, an amount of movement of the indicator surface within the dose display window when the force or touch is applied to the dose knob, wherein the amount of movement corresponds to an amount of medication injected.

14. The method of clause 13, further comprising transmitting the amount of medication injected to a receiver in wireless communication with the dose monitoring system.

15. The method of clause 13 or 14, further comprising displaying dose information on a display of the receiver.

16. The method of any of clauses 13 to 15, further comprising transitioning the injection detection assembly from a low power state to a full power state when the force or touch is detected by the injection sensor.

17. The method of any of clauses 13 to 16, further comprising transitioning the injection detection assembly from a low power state to a full power state when the injection sensor is pressed according to a predetermined activation pattern.

18. The method of any of clauses 13 to 17, further comprising determining a set dose based on an amount of movement of the dose indicator surface when no force or touch is applied to the injection sensor.

19. The method of clause 18, further comprising displaying the set dose on a display of the dose detection assembly.

20. The method of any of clauses 13 to 19, further comprising detecting relative movement of the dose detection assembly and the body of the injection pen by detecting a change in a position of one or more edges of the dose display window by the optical sensor assembly.

21. The method of clause 20, further comprising correcting the set dose or the injected dose based on the change in the position of the one or more edges of the dose display window.

22. Apparatus for detecting dose information by a dose monitoring system coupled to an injection pen, the apparatus comprising:
means for detecting movement of an indicator surface in a dose display window of an injection pen by an optical sensor assembly of a dose detection assembly of the dose monitoring system, wherein the dose detection assembly is secured to a body of the injection pen over the dose display window of the injection pen;
means for detecting a force or touch applied to an injection sensor of an injection detection assembly of the dose monitoring system, wherein the injection detection assembly is secured to a dose knob of the injection pen;
means for transmitting injection information from the injection detection assembly to the dose detection assembly when the force or touch is applied to the injection sensor; and
means for determining, by a processor of the dose monitoring system or dose detection assembly, an amount of movement of the indicator surface within the dose display window when the force or touch is applied to the dose knob, wherein the amount of movement corresponds to an amount of medication injected.

23. The apparatus according to clause 22 further comprising means for implementing the method according to any of clauses 13 to 21.

24. A dose monitoring system for an injection pen, the dose monitoring system comprising:
a housing configured to be secured to an injection pen over a dose display window of the injection pen;
a sensor assembly configured to determine an amount of movement of a dose indicator surface within the dose display window of the injection pen;
an injection sensor configured to detect a touch or force applied to a dose knob of the injection pen; and
a processor configured to determine an amount of medication injected based on the amount of movement of the dose indicator surface within the dose display window when the touch or force is applied to the dose knob.

25. The dose monitoring system of clause 24, further comprising a transceiver configured to communicate dose information.

26. The dose monitoring system of clause 24 or 25, wherein the processor is coupled to the housing.

27. The dose monitoring system of any of clauses 24 to 26, wherein the sensor assembly comprises an optical sensor assembly.

28. The dose monitoring system of clause 27, wherein the optical sensor assembly comprises an optical navigation sensor.

29. The dose monitoring system of any of clauses 24 to 28, wherein the housing comprises a securement device for securing the housing to the injection pen.

30. The dose monitoring system of clause 29, wherein the securement device comprises a bore.

31. The dose monitoring system of clause 29 or 30, wherein the securement device comprises one or more clips.

32. The dose monitoring system of any of clauses 24 to 31, wherein the housing comprises one or more locators configured to align the optical sensor assembly over the dose display window.

33. The dose monitoring system of any of clauses 24 to 32, wherein the processor is configured to execute a machine learning model to determine dose information.

34. The dose monitoring system of any of clauses 24 to 33, wherein the processor is configured to execute a machine learning model to determine an injection schedule of a user.

What is claimed is:

1. A dose monitoring system for an injection pen, the dose monitoring system comprising:
a dose detection assembly, comprising:
a housing configured to be secured to a body of the injection pen over a dose display window of the injection pen, and
an optical sensor assembly configured to determine an amount of movement of a dose indicator surface within the dose display window;
an injection detection assembly, comprising:
a body configured to be secured to a dose knob of the injection pen,
an injection sensor configured to detect a force or touch applied to the dose knob of the injection pen, and
a transmitter configured to communicate injection information when the injection sensor detects the force or touch applied to the dose knob; and
a processor in communication with the optical sensor and the injection sensor that is configured to determine an amount of medication injected based on the amount of movement of the dose indicator surface within the dose display window when the force or touch is applied to the dose knob as detected by the injection sensor.

2. The system of claim 1, wherein the dose detection assembly comprises a securement device configured to secure the dose detection assembly to the body of the injection pen.

3. The system of claim 1, wherein the dose detection assembly is configured to determine a set dose based on an amount of movement of the dose indicator surface within the dose display window as determined by the optical sensor assembly when no force or touch is detected by the injection sensor.

4. The system of claim 3, wherein the dose detection assembly further comprises a display configured to display the set dose.

5. The system of claim 3, wherein the dose detection assembly is configured to determine that the set dose was fully injected when the amount of movement within the dose display window when the force or touch is applied to the dose knob is equal to the amount of movement of the dose indicator surface when no force or touch was applied to the dose knob.

6. The system of claim 1, wherein the injection sensor of the injection detection assembly comprises a touch sensor.

7. The system of claim 1, wherein the optical sensor assembly comprises an optical navigation sensor.

8. The system of claim 1, wherein the optical sensor assembly comprises a light source configured to direct light at the dose indicator surface of the injection pen.

9. The system of claim 1, wherein the optical sensor assembly is configured to detect one or more edges of the dose display window.

10. The system of claim 9, wherein the processor is configured to detect relative movement of the dose detection assembly and the body of the injection pen based on detection of a position of the one or more edges of the dose display window.

11. The system of claim 1, wherein the transmitter of the injection detection assembly comprises a low power mode and a full power mode, wherein the injection detection assembly is configured to transition to the full power mode when the injection sensor is pressed.

12. A method for detecting dose information by a dose monitoring system coupled to an injection pen, the method comprising:
detecting movement of an indicator surface in a dose display window of an injection pen by an optical sensor assembly of a dose detection assembly of the dose monitoring system, wherein the dose detection assembly is secured to a body of the injection pen over the dose display window of the injection pen;
detecting a force or touch applied to an injection sensor of an injection detection assembly of the dose monitoring system, wherein the injection detection assembly is secured to a dose knob of the injection pen;
transmitting injection information from the injection detection assembly to the dose detection assembly when the force or touch is applied to the injection sensor; and
determining, by a processor of the dose monitoring system, an amount of movement of the indicator surface within the dose display window when the force or touch is applied to the dose knob, wherein the amount of movement corresponds to an amount of medication injected.

13. The method of claim 12, further comprising transmitting the amount of medication injected to a receiver in wireless communication with the dose monitoring system.

14. The method of claim 13, further comprising displaying dose information on a display of the receiver.

15. The method of claim 12, further comprising transitioning the injection detection assembly from a low power state to a full power state when the force or touch is detected by the injection sensor.

16. The method of claim 15, further comprising transitioning the injection detection assembly from a low power state to a full power state when the injection sensor is pressed according to a predetermined activation pattern.

17. The method of claim 12, further comprising determining a set dose based on an amount of movement of the dose indicator surface when no force or touch is applied to the injection sensor.

18. The method of claim 17, further comprising displaying the set dose on a display of the dose detection assembly.

19. The method of claim 12, further comprising detecting relative movement of the dose detection assembly and the body of the injection pen by detecting a change in a position of one or more edges of the dose display window by the optical sensor assembly.

20. The method of claim 19, further comprising correcting the set dose or the injected dose based on the change in the position of the one or more edges of the dose display window.
